(19) United States
(12) Patent Application Publication
(10) Pub. No.: US 2003/0134744 A1
(43) Pub. Date: Jul. 17, 2003

(54) Process for production and distribution of a prereduced selective hydrogenation catalyst

(75) Inventors: Steven A. Blankenship, Radcliff, KY (US); Jennifer A. Perkins, Crestwood, KY (US); Andrzej Rokicki, Prospect, KY (US); James E. Fried JR., Buckner, KY (US)

Correspondence Address: Scott R. Cox, Suite 2200, 400 West Market St., Louisville, KY, 40202, US

(73) Assignee: Sud-Chemie Inc., Louisville, KY

(21) Appl. No.: 10/025,663

(22) Filed: Dec. 19, 2001

(51) Int. Cl. B01J 23/42; C10G 45/00; C07C 7/163; B01J 23/44

(52) U.S. Cl. 502/339; 208/143; 585/258; 585/259

(57) ABSTRACT

A process for preparation of a selective hydrogenation catalyst including preparing a catalyst material containing palladium and preferably additional additive materials, prereducing the palladium material and the additional additive materials, storing the prereduced catalyst under a non-oxidizing material and distributing the prereduced catalyst in a shipping container under the non-oxidizing material to a customer for use in a selective hydrogenation reaction.

### CROSS REFERENCE TO RELATED APPLICATIONS

NONE

### BACKGROUND OF INVENTION

1. Field of Invention

This invention relates to a process for the production and distribution of a prereduced selective hydrogenation catalyst for use in an olefinic feed stream. This invention also relates to a process of use of a prereduced hydrogenation catalyst for the selective hydrogenation of an olefinic feed stream.

2. Prior Art

The manufacture of unsaturated hydrocarbons usually involves cracking various types of hydrocarbons and often produces a crude product containing hydrocarbon impurities that are more unsaturated than the desired product. These unsaturated hydrocarbon impurities are often very difficult to separate by fractionation from the desired product. A common example of this problem occurs with ethylene purification, in which acetylene is a common impurity. It is often difficult, industrially, to remove such undesirable, highly unsaturated hydrocarbons without significant hydrogenation of the desired hydrocarbons. One example of this process is described in UK Pat. No. 916,056.

Two general types of gas phase selective hydrogenation processes for removing undesired, unsaturated hydrocarbons have come into use. One, known as “front-end” hydrogenation, involves passing the crude gas from the initial cracking step, after removal of steam and condensible organic material, over a hydrogenation catalyst. Despite the large hydrogen content of such gas, which is very greatly in excess of the quantity of acetylenes that are present and which quantity should be sufficient to hydrogenate a substantial part of those acetylenes, substantially complete hydrogenation of the acetylenes with sufficient selectivity to produce olefins of polymerization quality is often a problem. The high concentration of hydrogen present in the front-end systems requires a very selective catalyst that does not also substantially hydrogenate the ethylene that is also present in the feed stream. Overhydrogenation can lead to thermal excursion known as “run-away”. Under “run-away” conditions, high temperatures are experienced, severe loss of ethylene occurs and catalyst damage takes place. In addition, furnace upsets in the front-end reactor system can result in swings of CO concentration from moderate levels to very low levels. Existing front-end catalysts cannot tolerate these substantial swings in CO concentration very well and often are prone to “run-away”. In a front-end reactor system, the catalyst is also exposed to high space velocity operations of about 10,000-12,000 GHSV per bed.

In the other type of gas phase selective hydrogenation, known as “tail-end” hydrogenation, the crude gas is fractionated and the resulting concentrated product streams are individually reacted with hydrogen in a slight excess over the quantity required for hydrogenation of the unsaturated acetylenes which are present. Tail-end reactor systems operate at a GHSV of 2500-5000 per bed. In tail-end hydrogenation there is a greater tendency for deactivation of the catalyst during the hydrogenation procedure, and consequently, periodic regeneration of the catalyst is necessary. While the amount of hydrogen and carbon monoxide addition can be adjusted to maintain selectivity, formation of polymers is a major problem.

A number of patents have discussed the selective hydrogenation of unsaturated hydrocarbons such as U.S. Pat. Nos. 4,126,645, 4,367,353, 4,329,530, 4,347,392 and 5,414,170.

The catalysts that are preferred for selective hydrogenation reactions generally comprise palladium supported on an alumina substrate, as disclosed, for example, in U.S. Pat. Nos. 3,113,980, 4,126,645 and 4,329,530. Other gas phase palladium on alumina catalysts for the selective hydrogenation of acetylene compounds are disclosed, for example, in U.S. Pat. Nos. 5,925,799, 5,889,138, 5,648,576 and 4,126,645.

One of the problems that frequently occurs with palladium on alumina catalysts is that under normal operating conditions not only is the acetylene hydrogenated, a substantial portion of the ethylene is also converted to ethane. In addition, these palladium on alumina catalysts often have relatively low stability over extended periods of use due to the formation of large quantities of oligomers on the catalyst surface. To overcome this problem, enhancers or additives are often added to the palladium catalyst to improve its performance. One common additive is silver. For example, acetylene hydrogenation catalysts for ethylene purification comprising palladium and silver on a support material are disclosed in U.S. Pat. Nos. 4,404,124, 4,484,015, 5,488,024, 5,489,565 and 5,648,576. In one specific example U.S. Pat. No. 5,648,576 discloses a selective hydrogenation catalyst for acetylene compounds comprising from about 0.01 to 0.5 weight percent of palladium and from about 0.001 to 0.02 percent by weight of silver. 80 percent or more of the silver is placed within a thin layer near the surface of the carrier body.

Catalysts comprising palladium, silver, an alkali metal fluoride and a support material, which are utilized for the hydrogenation of other feed stream impurities, such as dienes and diolefins, are disclosed, for example, in U.S. Pat. No. 5,489,565.

Catalysts comprising palladium and gold on a catalyst support which may be used for the hydrogenation of acetylenes and diolefins have been suggested by U.S. Pat. Nos. 4,533,779 and 4,490,481. These patents disclose the use of a substantially greater amount of palladium than of gold, specifically 0.03 to about 1 percent by weight palladium and from 0.003 to 0.3 percent by weight gold.

While conventional palladium or silver/palladium catalysts for the selective hydrogenation of acetylene have been useful, there are still a number of problems encountered when they are used, including a relatively low tolerance to carbon monoxide concentration swings, lower selectivity than is desirable by the industry, and problems with high space velocity operations.

The manufacturing process for silver and palladium hydrogenation catalysts generally includes reduction of the metallic oxides to their elemental states. However, because the silver and the palladium on these promoted catalysts reoxidize quite easily during conventional preparation, transportation, installation and use, for optimum performance it is necessary to again reduce the palladium and palladium/silver in the promoted catalysts in situ before selective hydrogenation of the acetylene can occur. Because hydrogen pre-reduction in situ is not readily available in most commercial plants, catalyst activation with feed stock is the most common method of reduction in situ.

In the typical process for the preparation of a hydrogenation catalyst, particularly a palladium or silver/palladium catalyst, a carrier material, such as alpha alumina, is impregnated with a palladium compound, such as palladium chloride and, when silver is used as an additive, a silver compound such as silver nitrate. See, for example, U.S. Pat. No. 4,404,124.

The impregnated catalyst precursor material is then dried. While the material may then be used directly as a catalyst for hydrogenation, it is generally reduced prior to the drying step, often by wet reduction. After wet reduction the catalyst is washed to remove halides and dried. This drying step, which is normally conducted under air, generally reoxidizes the palladium and/or palladium/silver on the catalyst. After drying the catalyst is packaged and shipped to the customer without further processing. Thus, before the catalyst can be utilized for selective hydrogenation, the metallic oxides must be reduced in situ. For this in situ reduction step to be successful, the feed for the selective hydrogenation process must generally be modified from a conventional feed. Conventionally, the reduction step requires an increase in the amount of hydrogen which is present in the feed stream.

The industry has determined that reduction of the hydrogenation catalyst in situ with feed stock is an acceptable procedure which avoids the expenses associated with installing costly hydrogen reduction facilities. Processes for the reduction of the catalyst in situ are disclosed, for example, in U.S. Pat. Nos. 4,329,530, 4,577,047, 4,551,443, 4,404,124, 4,410,455 and 4,577,047. See also U.S. Pat. No. 5,955,397. Thus, the recognized process for the reduction of the active metal or metals on a selective hydrogenation catalyst is at the plant in situ where the selective hydrogenation process is conducted.

Difficulties are often experienced in this in situ reduction process during normal operations. It has been discovered that the normal temperature of the feed stream is not generally high enough to effectively reduce metal oxides that are present on prior art catalysts. In addition, the presence of carbon monoxide in a front end ethylene purification feed stream inhibits the in situ activation of the catalyst, thus necessitating a higher temperature for the feed to successfully hydrogenate the appropriate materials. Such higher temperatures reduce the performance of the catalyst and reduce its life expectancy. That the presence of carbon monoxide in the feed stream would inhibit reduction of the selective hydrogenation catalysts is surprising as carbon monoxide is often utilized as a reducing agent.

An additional problem with in situ reduction is that many of the existing plant reactors are not fitted with the equipment necessary to perform effective in situ activation of the catalyst prior to the introduction of the feed stock. Therefore, the feed stock must be utilized to reduce the catalyst. Because the catalyst has not yet been reduced when the feed stock initially contacts the catalyst, there is a reduction in the performance of that catalyst until sufficient hydrogen has passed over the catalyst to reduce the metal oxides located on the catalyst. Thus, in situ reduction is frequently inefficient, resulting in substandard performance of the catalyst.

Processes have been disclosed for the off site, wet reduction of catalyst material, for example as disclosed in U.S. Pat. No. 4,367,167. However, these off site, wet reduction processes ultimately result in unreduced catalysts because the wet reduced catalysts must be dried before they can be used in situ. As drying of the wet reduced catalyst is commonly conducted in air, the metals on the catalyst frequently reoxidize.

The processes of the invention are designed to address these problems and deficiencies in conventional catalytic hydrogenation reactions.

Accordingly, it is an object of this invention to disclose a process for the production of a catalyst for the selective hydrogenation of an olefinic feed stream containing acetylenic impurities.

It is a still further object of this invention to disclose a process for the production of a catalyst for the front-end and tail-end selective hydrogenation of acetylenic impurities, whereby the quantity of the desirable C₂ and C₃ olefins is not substantially reduced.

It is a still further object of this invention to disclose a process for the production of a catalyst for the front end and tail end selective hydrogenation of a C₂ and C₃ olefinic feed stream containing acetylenic impurities even when the quantity of carbon monoxide in the feed stream is high.

It is a still further object of the invention to disclose a catalyst for use in the selective hydrogenation of acetylenic impurities which is reduced prior to shipment to the end user.

It is a further object of the invention to disclose a catalyst for selective hydrogenation of acetylenic impurities prepared by an ex situ reduction process, whereby the temperature of reduction is controlled.

It is a still further object of the invention to disclose an ex situ reduced palladium-based selective hydrogenation catalyst for the selective hydrogenation of acetylene which exhibits enhanced selectivity and reduced polymer formation over conventional palladium-based selective hydrogenation catalysts in front-end and tail-end reactor systems.

It is a further object of the invention to disclose a process for the production of palladium and palladium/silver catalysts for the selective hydrogenation of acetylene, wherein the palladium and/or palladium and silver on the catalysts are reduced ex situ.

It is a still further object of the invention to disclose a process for the ex situ reduction of palladium and palladium/silver selective hydrogenation catalysts useful for the selective hydrogenation of acetylene, which catalysts exhibit enhanced selectivity, resistance to run-away, tolerance to CO concentration swings and improved performance at high gas hourly space velocity over conventional palladium and palladium/silver selective hydrogenation catalysts.

These and other objects can be obtained by the processes for the preparation of an ex situ, reduced selective hydrogenation catalysts for use in a C₂ and C₃ olefinic feed streams containing acetylenic impurities which is disclosed by the present invention.

### SUMMARY OF THE INVENTION

The present invention is a process for the production and distribution of a catalyst for the selective hydrogenation of acetylenic impurities in an olefinic feed stream comprising

preparing a carrier material in a suitable shape;

impregnating the carrier material with a palladium compound;

calcining the carrier material impregnated with the palladium compound;

prereducing the palladium compound to a metallic state to form a palladium catalyst;

packaging the prereduced palladium catalyst under a non-oxidizing material in a storage container; and

distributing the prereduced palladium catalyst contained in a storage container to a customer for use in a process for selective hydrogenation of the olefinic feed stream, whereby the prereduced palladium catalyst is not again reduced prior to utilization on stream.

The present invention further comprises a prereduced palladium catalyst for hydrogenation prepared by the process described above. The catalyst of the present invention may also include silver as an additive.

The invention further comprises a process for the selective hydrogenation of acetylenic impurities contained in an olefinic feed stream comprising passing the feed stream, which contains the acetylenic impurities, over a prereduced catalyst prepared by the process described above.

### DETAILED DESCRIPTION

The invention is a process for the production of a prereduced catalyst for selective hydrogenation. The invention is also a process for selective hydrogenation of a feed stream using the prereduced catalyst of the invention. The invention further comprises a catalyst produced by the process of the invention that is useful for selective hydrogenation. The catalyst of the invention is designed primarily for selective hydrogenation procedures, preferably of acetylene in admixture with ethylene.

A front end reactor feed stream for such selective hydrogenation procedures normally includes substantial quantities of hydrogen, methane, ethane, ethylene, carbon monoxide and carbon dioxide, as well as various impurities, such as acetylene. The goal of selective hydrogenation is to reduce substantially the amount of the acetylene present in the feed stream without substantially reducing the amount of ethylene that is present. If substantial hydrogenation of the ethylene occurs, thermal run-away can also occur.

The catalyst prepared by the process of the invention exhibits improved selectivity, resistance to run-away, tolerance to CO concentration swings and improved performance at higher gas hourly space velocities (GHSV) over prior art selective hydrogenation catalysts. In addition to utilization for front-end purification, the catalyst of the invention is also useful for tail-end ethylene purification where the catalysts exhibit improved selectivity and reduced polymer formation. The process of prereduction of the catalyst ex situ is critical to the enhanced performance of hydrogenation catalysts of the invention.

The catalyst that is useful for this improvement in the selective hydrogenation process is comprised of a catalyst carrier onto which palladium is impregnated. In addition to palladium, other metals such as silver, tin copper, gold, lead, thallium, bismuth, cerium and alkali metals may be added to the catalyst as additives. Preferably one or more additives are added to the catalyst which are selected from silver, alkali metals, gold and thallium. The most preferred additive utilized is silver. These additives may be introduced to the catalyst by conventional procedures.

The catalyst carrier may be formed of any catalyst carrier material with a surface area less than about 250 m²/g, such as alumina, zinc oxide, nickel spinel, titania, magnesium oxide and cerium oxide. In a preferred embodiment, the catalyst carrier is formed from alpha alumina. The surface area of the catalyst carrier is preferably from about 1 to about 250 m²/g and more preferably from about 1 to about 75 m²/g. Its pore volume is preferably from about 0.2 to about 0.7 cc/g. The catalyst carrier can be formed in any suitable size and shape. Preferably it is formed as particles from about 2 to about 6 millimeters in diameter, which are formed into shapes, such as spherical, cylindrical, trilobel and the like. In a more preferred embodiment the catalyst carrier is formed in a spherical shape.

The palladium can be added to the catalyst carrier by any conventional procedure. The presently preferred procedure requires impregnating the catalyst carrier with an aqueous solution of a palladium salt, such as palladium chloride or palladium nitrate, preferably palladium chloride. The extent of penetration of the palladium into the carrier can be controlled by adjustment of the pH of the solution. In a preferred embodiment, the depth of penetration of the palladium salt is controlled such that approximately 90 percent of the palladium salt is contained within 250 microns of the surface of the catalyst carrier. Any suitable method can be used to achieve the preferred palladium penetration, such as is disclosed in U.S. Pat. Nos. 4,484,015 and 4,404,124. After palladium impregnation, the impregnated catalyst composition is calcined at a temperature from about 400 to about 600 degrees C. for about one hour.

Once the palladium-impregnated catalyst composition has been calcined, additives may be added to the catalyst. In one preferred embodiment the additional additive is a metallic additive, preferably an alkali metal, gold, silver and/or thallium additive, and most preferably a silver additive which is impregnated in the form of a salt solution. For example, when silver is utilized the preferred salt is silver nitrate. The palladium/metallic additive impregnated catalyst material is then calcined at a temperature from about 400 to about 600 degrees C. for about one hour.

In an alternative embodiment the additive material and the palladium salt can be co-impregnated and calcined.

The amount of the palladium present after drying is preferably from about 0.001 to about 0.028 weight percent, more preferably 0.01 to about 0.02 weight percent, based on the total weight of the catalyst. When silver is used as an additive, the amount of silver present on the catalyst after drying is preferably from about 0.04 to about 1.0 percent, more preferably 0.04 to 0.12 weight percent based on the total weight of the catalyst. The ratio of the silver to palladium on a by-weight basis is preferably from about 2.1 to about 20.1, more preferably 2:1 to about 6.1, and most preferably from about 12:1 to about 20:1. It is preferred to employ an aqueous silver nitrate solution in a quantity greater than is necessary to fill the pore volume of the catalyst.

The metals contained in the palladium or metal additive/palladium catalyst precursor are then reduced. To reduce the catalyst, it is treated with hydrogen during a heating step. The temperature of this heating step is from about 200 to about 1000° F. (93 to about 537° C.), preferably 200 to 900° F. (93 to about 482° C.). The catalyst is heated at the preferred temperature for about 1 to 5 hours, preferably 1 to 3 hours.

Following drying and reducing of the catalyst, it is important that the reduced catalyst be stored under a non-oxidizing atmosphere to prevent reoxidation. The term “non-oxidizing atmosphere” refers to gases which do not react with the species present in the reaction environment to reoxidize the metals. The preferred non-oxidizing gases include carbon dioxide, nitrogen, helium, neon, and argon with carbon dioxide and nitrogen more preferred. Air and oxygen are not appropriate because they reoxidize or deactivate the hydrogenation catalyst. Once the reduced catalyst is placed under a non-oxidizing gas, it is loaded into individual containers. The individual containers are then purged with the same or a different non-oxidizing gas and sealed to prevent contact of the catalyst material with a reoxidizing environment. The sealed catalyst container is then ready for shipment to the reactor site for loading into the reactor. In one example the reduced catalyst is loaded in a conventional container under carbon dioxide or nitrogen. The container is then wrapped securely with a plastic wrap material that is air impermeable.

In use, the catalyst is placed in a reactor and the selective hydrogenation reaction is immediately begun. By use of the catalyst of the invention, it is not necessary to reduce the catalyst in situ before hydrogenation of the compounds in the feed stream. Accordingly, selective hydrogenation of compounds, such as a acetylene, can immediately begin. Such selective hydrogenation occurs when a gas stream containing primarily hydrogen, ethylene, acetylene and carbon monoxide is passed over the catalyst of the invention. During this process the inlet temperature of the feed stream is raised to a level sufficient to hydrogenate the acetylene. Generally, this temperature range is from about 35° C. to about 100° C. Any suitable reaction pressure can be used. Generally, the total pressure is in the range of about 100 to 1000 psig (700+7000 KPa) with the gas hourly space velocity (GHSV) in the range of about 1000 to about 14000 liters per liter of catalyst per hour.

It has been surprisingly discovered that the prereduced catalyst of the invention performs better than a catalyst with a similar composition which is activated in situ under feed stock. For example, it has been surprisingly discovered that catalysts which are reduced in hydrogen ex situ and then shipped to the reactor under a non-oxidizing gas have a higher selectivity and better activity than catalysts which are merely activated in situ with feed stock.

It has also been surprisingly discovered that the prereduced catalyst of the invention performs better than a conventional catalyst which is activated with feed, especially when the feed stream has a relatively high concentration of carbon monoxide. Further, it has been surprisingly discovered that the prereduced catalyst of the invention performs better than catalysts with a similar composition which are activated under feed for both front-end and tail-end hydrogenation reactions. An important feature of the invention is the ability of the prereduced catalyst to perform well under high GHSV condition, as high as 12,000 GHSV. Conventional catalysts reduced under feed do not perform as well under these conditions.

### EXAMPLES

### Example 1—Invention

A commercial catalyst was acquired from Süd-Chemie Inc. with a product name of G83A. It comprised an alumina carrier onto which a palladium additive had been added and contained approximately 0.018 percent by weight palladium and about 99 percent weight alumina. It had a BET surface area of 3.7 m²/g. Approximately 25 ccs of the catalyst were placed in a catalyst bed which was purged with nitrogen.

The catalyst bed was gradually heated to 200° F. (93° C.). Once this temperature was reached, the nitrogen was discontinued and hydrogen was introduced into the chamber for at least 60 minutes to reduce the catalyst. Upon completion of the reduction cycle, nitrogen was again introduced into the bed and it was cooled to room temperature. The reduced catalyst was kept under nitrogen atmosphere and loaded into an individual container. This container was purged with nitrogen gas and then sealed to prevent contact with air until it was tested.

### Comparative Example 2

A catalyst with the same composition as the catalyst of Example 1 was acquired from Süd-Chemie Inc. It was not reduced prior to testing.

### Example 3—Invention

A commercial catalyst designated as G83C was acquired from Süd-Chemie Inc. It was a palladium catalyst onto which silver had been added as an additive. It contained 0.018 weight percent palladium and 0.07 weight percent silver on an alumina carrier. It had a BET surface area of about 3.7 m²/g. The catalyst was placed in a bed and purged with nitrogen while the bed was heated to 200° F. (93° C.). Once that temperature was reached, the nitrogen was discontinued and hydrogen was introduced into the reaction chamber for at least 60 minutes to reduce the catalyst. Upon completion of the reduction cycle, nitrogen was introduced into the bed as it was cooled to room temperature. The reduced catalyst was loaded into an individual container and kept under a nitrogen atmosphere. The container was purged with nitrogen gas and then sealed to prevent contact with air until it was tested.

### Comparative Example 4

An additional quantity of the catalyst material of Example 3 was acquired. However, it was not reduced in the manner of Example 3 prior to testing.

### Example 5—Invention

A reduced catalyst was prepared in the same manner as described in Example 3 except the purging gas was carbon dioxide rather than nitrogen.

### Example 6—Invention

A commercial catalyst designated as G58D was acquired from Süd-Chemie Inc. It was a palladium catalyst containing a silver additive. This catalyst contained 0.018 weight percent palladium and 0.012 weight percent silver on an alumina carrier and had a BET surface area of about 3.7 m²/g. The catalyst was reduced and placed in a sealed container under nitrogen in the same manner as described in Example 1, except it was reduced at a temperature of 100° F. (38° C.).

### Example 7—Invention

The same process as described in Example 6 was conducted on another sample of the catalyst of Example 6 except the temperature of reduction was 150° F. (65° C.).

### Example 8—Invention

The same process as described in Example 6 was conducted on another sample of the catalyst of Example 6 except the temperature of reduction was 200° F. (93° C.).

### Example 9—Invention

The same process as described in Example 6 was conducted on another sample of the catalyst of Example 6 except the temperature of reduction was 400° F. (204° C.).

### Example 10—Invention

The same process as described in Example 6 was conducted on another sample of the catalyst of Example 6 except the temperature of reduction was 700° F. (371° C.).

### Comparative Example 11

Another sample of the same catalyst as was used in Examples 6-10 was acquired. However, it was not reduced prior to testing.

### Example 12—Invention

A palladium/silver catalyst on alumina carrier designated as G58E was acquired from Süd-Chemie Inc. It contained 0.047 weight percent palladium and 0.282 weight percent silver on an alumina carrier and had a BET surface area of about 150 m²/g. The catalyst was reduced and placed in a sealed container in the same manner as described in Example 1 except the temperature of reduction was 140° F. (60° C.).

### Example 13—Invention

The same catalyst as in Example 12 was reduced using the same process as disclosed in Example 12 except that it was reduced for 3 hours at 400° F. (204° C.).

### Example 14—Invention

The same catalyst as in Example 12 was reduced using the same process as disclosed in Example 12 except that it was reduced for 3 hours at 600° F. (315° C.).

### Example 15—Invention

The same catalyst as in Example 12 was reduced using the same process as disclosed in Example 12 except that it was reduced for 3 hours at 800° F. (427° C.)

### Comparative Example 16

Another sample of the same catalyst as was used in Examples 12-15 was acquired. However, it was not reduced prior to testing.

### TABLES

### Table 1

The catalysts of inventive Examples 1, 3 and 5 and Comparative Examples 2 and 4 were tested using a laboratory simulated feed stream in a front-end ethylene purification reactor that employed de-ethanizer separation technology in front of the selective hydrogenation reactor. A moderate GHSV space velocity of 7000 was used at a pressure of 500 psig (3500 KPa) 25 ccs of the catalyst sample were placed in a catalyst bed for testing. The catalyst sample was evaluated in a bench scale ¾ in. I.D. reactor tube. Simulated process feed streams were prepared for catalyst evaluation. The feed streams comprised 1 percent C₂H₆, 45 percent C₂H₄, 2800 ppm C₂H₂, 20 percent H₂ and 250 to 300 ppm CO with the remaining gas comprising CH₄. The catalysts were tested for 8 hours. Temperature was gradually increased, starting at 87° F. (30.5° C.). Data was taken every twenty minutes at 4° F. (2° C.) intervals as the temperature increased. The clean-up temperature (T₁) when exit C₂H₂ level was <25 ppm was noted. The temperature was increased past T₁ until “runaway” occurred (T₂), i.e. when >4% hydrogen loss occurred. This temperature minus T₁ was a measure of the selectivity of the catalyst. Higher T₂−T₁ indicates greater selectivity and better thermal stability. The results of the testing are shown in the following Table.

| | | T₁ | T₂ | | Selectivity |
|---|---|---|---|---|---|
| Run | Catalyst | (° F.) | (° F.) | T₂-T₁ | at T₁ |
| Example 1 | G83A (SCI) Pd/Al₂O₃ | 114 | 128 | 14 | 11% |
| (Invention) | Reduced in 100% H₂ | | | | |
| | at 200° F. | | | | |
| | (93° C.) for 1 hour, | | | | |
| | stabilized in N₂ | | | | |
| Comparative | G83A (SCI) Pd/Al₂O₃ | 140 | 150 | 10 | 3% |
| Example 2 | | | | | |
| Example 3 | G83C (SCI) | 106 | 124 | 18 | 31.9% |
| (Invention) | Pd/Ag/Al₂O₃ Reduced | | | | |
| | in 100% H₂ at 200° F. | | | | |
| | (93° C.) for 1 hour, | | | | |
| | stabilized in N₂ | | | | |
| Comparative | G83C (SCI) | 103 | 107 | 4 | −174.7% |
| Example 4 | Pd/Ag/Al₂O₃ | | | | |
| Example 5 | G83C (SCI) | 106 | 122 | 16 | −24.8% |
| (Invention) | Pd/Ag/Al₂O₃ Reduced | | | | |
| | in 100% H₂ at 200° F. | | | | |
| | (93° C.) for 1 hour, | | | | |
| | stabilized in CO₂ | | | | |

This data clearly shows that the catalysts of the invention (Examples 1, 3 and 5) have greater selectivity than the catalysts of Comparative Example 2. (The greater the value of the T₂−T₁, the greater the selectivity of the catalyst.) Examples 3 and 5 also exhibited higher selectivity than Comparative Example 4 as shown by the greater value for T₂−T₁.

### Table II

This Table shows the performance of the catalyst of the invention under higher GHSV conditions. A de-ethanizer feed was tested at a space velocity of 12000 GHSV. The feed contained the same composition of feed gases as was present in Table I.

| | | T₁ | T₂ | | Selectivity |
|---|---|---|---|---|---|
| Run | Catalyst | (° F.) | (° F.) | T₂-T₁ | at T₁ |
| Example 1 | G83A (SCI) Pd/Al₂O₃ | 124 | 136 | 12 | −40% |
| (Invention) | Reduced in 100% H₂ | | | | |
| | at 200° F. (93° C.) | | | | |
| | for 1 hour, | | | | |
| | stabilized in N₂ | | | | |
| Comparative | G83A (SCI) Pd/Al₂O₃ | 143 | 148 | 5 | −123% |
| Example 2 | | | | | |
| Example 3 | G83C (SCI) | 126 | 135 | 9 | −11.6% |
| (Invention) | Pd/Ag/Al₂O₃ Reduced | | | | |
| | in 100% H₂ at 200° F. | | | | |
| | (93° C.) for 1 hour, | | | | |
| | stabilized in N₂ | | | | |
| Comparative | G83C (SCI) Pd/Al₂O₃ | N/A | 124 | — | — |
| Example 4 | | | | | |

This Table clearly shows a greater selectivity and stability of the inventive example, Example 1, over the comparative example, Comparative Example 2. The non-reduced catalyst could not significantly remove C₂H₂ from the feed stream under these conditions. The lower temperature of T₁ obtained with Example 1, in comparison to Comparative Example 2 also indicates a higher activity level, as shown by the lower clean-up temperature for the inventive catalyst. The non-reduced silver-promoted catalyst of Example 4 could not significantly remove C₂H₂ from the feed stream under these conditions. The silver-promoted catalyst of Example 3 successfully removed acetylene even at this high space velocity.

### Table III

The purpose of this Table is to show the performance of the catalyst of the invention with different types of feed stock, particularly with a high carbon monoxide concentration. The feed stream contained 1 percent C₂H₆, 18 percent C₂H₄, 14 ppm C₂H₂, 20 percent H₂, 3 percent C₃H₆, 0.02 percent C₃H₈, 8060 ppm CO and the remaining portion CH₄.

| | | T₁ | T₂ | | Selectivity |
|---|---|---|---|---|---|
| Run | Catalyst | (° F.) | (° F.) | T₂-T₁ | at T₁ |
| Example 1 | G83A (SCI) Pd/Al₂O₃ | 140 | 161 | 21 | 7% |
| (Invention) | Reduced in 100% H₂ | | | | |
| | at 200° F. (93° C.) for | | | | |
| | 1 hour, stabilized in | | | | |
| | N₂ | | | | |
| Comparative | G83C (SCI) Pd/Al₂O₃ | 151 | 159 | 8 | −28% |
| Example 2 | | | | | |

As can be seen from Table III, the catalyst of the invention (Example 1) outperformed the currently available non-reduced comparative catalyst (Comparative Example 2) by exhibiting higher selectivity and stability (T₂−T₁). (The higher activity for the invention is measured by the lower value of T₁.) This is especially impressive considering the high quantity of CO present (8060 ppm).

### Table IV

Table IV shows another example of the performance of the catalyst of the invention, both with and without the addition of silver as a promoter. The feed stream is contained in a front-end reactor system utilizing deproponizer separation before the C₂H₂ reactor. The feed contained 21 percent CH₄, 1 percent C₂H₆, 53 percent C₂H₄, 0.03 percent C₃H₈, 6 percent C₃H₆, 0.05 percent propadiene, 0.044 percent C₂H₂, 0.16 percent methylacetylene, 18.5 percent H₂, 0.05 percent CO and the remaining portion CH₄.

| | | T₁ | T₂ | | Selectivity |
|---|---|---|---|---|---|
| Run | Catalyst | (° F.) | (° F.) | T₂-T₁ | at T₁ |
| Example 1 | G83A (SCI) Pd/Al₂O₃ | 100 | 156 | 56 | 72% |
| (Invention) | Pre-reduced in 100% | | | | |
| | H₂ at 200° F. | | | | |
| | (93° C.) for 1 hour, | | | | |
| | stabilized in N₂ | | | | |
| Comparative | G83A (SCI) Pd/Al₂O₃ | 136 | 166 | 30 | 51% |
| Example 2 | | | | | |
| Example 3 | G83C (SCI) | 126 | 154 | 28 | −85% |
| (Invention) | Pd/Ag/Al₂O₃ Pre- | | | | |
| | reduced in 100% H₂ at | | | | |
| | 200° F. (93° C.) for 1 | | | | |
| | hour, stabilized in N₂ | | | | |
| Comparative | G83C (SCI) | 127 | 147 | 20 | −290% |
| Example 4 | Pd/Ag/Al₂O₃ | | | | |

As can be seen from Table IV, the catalyst of the invention (Example 1) outperformed the non-reduced comparative catalyst (Comparative Example 2) in selectivity (higher T₂−T₁) and stability. The lower value of T₁ of the inventive Example 1 indicates higher activity.

### Table V

The purpose of Table V is to show the impact of different temperatures of reduction on the performance of the various catalysts. The feed stream is comprised of a de-ethanizer feed under 7000 GHSV consisting of one percent C₂H₆, 45 percent C₂H₄, 2800 ppm C₂H₂, 20 percent H₂, 250-300 ppm CO and the remaining portion CH₄.

| | | T₁ | T₂ | |
|---|---|---|---|---|
| Run | Catalyst | (° F.) | (° F.) | T₂-T₁ |
| Example 6 | G58D | 123 | 141 | 18 |
| (Invention) | 100% hydrogen reduction at | | | |
| | 100° F. (38° C.) | | | |
| Example 7 | G58D | 127 | 141 | 14 |
| (Invention) | 100% hydrogen reduction at | | | |
| | 150° F. (68° C.) | | | |
| Example 8 | G58D | 137 | 149 | 12 |
| (Invention) | 100% hydrogen reduction at | | | |
| | 200° F. (93° C.) | | | |
| Example 9 | G58D | 127 | 140 | 13 |
| (Invention) | 100% hydrogen reduction at | | | |
| | 400° F. (204° C.) | | | |
| Example 10 | G58D | N/A* | 147 | N/A* |
| (Invention) | 100% hydrogen reduction at | | | |
| | 700° F. (371° C.) | | | |
| Comparison | G58D | 103 | 110 | 7 |
| Example 11 | no hydrogen reduction | | | |

This Table shows that the performance of the catalysts of the invention (Examples 6-10) is better than that of a catalyst which is not prereduced (Comparison Example 11). The optimized performance was present in Example 6 which was prereduced at 100° F. (38° C.).

### Table VI

The process used for the production of the catalyst of the invention is also useful for tail-end purification as is shown in the Table VI. The tail-end feed was comprised of 1 percent C₂H₂, 1.5 percent H₂ with the balance being C₂H₄. The catalysts of the invention were prereduced at various temperatures and over various times under a space velocity of 5000 GHSV.

| | | % C₂H₂ | % | Polymer |
|---|---|---|---|---|
| Run | Catalyst | conversion | Selectivity | formed |
| Example 12 | G58E Reduced in | 97.3 | 38.8 | 0.9430 |
| (Invention) | 100% H₂ for 1 hour | | | |
| | at 140° F. (60° C.) | | | |
| Example 13 | G58E | 94.7 | 40.4 | 0.4419 |
| (Invention) | Reduced in 100% H₂ for | | | |
| | 3 hours at 400° F. | | | |
| | (204° C.) | | | |
| Example 14 | G58E | 94.4 | 39.0 | 0.2907 |
| (Invention) | Reduced in 100% H₂ for | | | |
| | 3 hours at 600° F. | | | |
| | (315° C.) | | | |
| Example 15 | G58E | 95.4 | 48.8 | 0.3759 |
| (Invention) | Reduced in 100% H₂ for | | | |
| | 3 hours at 800° F. | | | |
| | (427° C.) | | | |
| Comparative | G58E | 92.1 | 21.1 | 0.9414 |
| Example 16 | | | | |

Upon review of the Table, it is clear that the catalyst of the invention (Examples 12-15) showed improved performance in situ as each had a higher selectivity compared to the commercially available catalyst of Comparative Example 16. A noticeable reduction in polymer formation was also evidenced in inventive Examples 13-15.

In addition, each of the examples showed that the catalysts produced by the process of the invention which was prereduced performed better than catalysts activated with feed stock in situ even when there was substantial hydrogen and carbon monoxide present in the in situ feed stream.

It will be apparent from the foregoing that while particular forms of the invention have been illustrated, various modifications can be made without departing from the scope of the invention.

1. A process for the manufacture of a catalyst for selective hydrogenation of a feed stock comprising
preparing a catalyst support,
impregnating the catalyst support with a palladium metal source,
reducing the palladium-impregnated catalyst with a reducing material,
without permitting the reduced catalyst to reoxidize, placing the reduced catalyst in a container under a non-oxidizing material, and
distributing the prereduced catalyst in the container to a customer while maintaining the reduced catalyst under the non-oxidizing material.

2. The process of claim 1 wherein the temperature of reduction of the catalyst is from about 50° F. to about 1000° F. (10° C. to 538° C.).

3. The process of claim 1 wherein the non-oxidizing material is selected from the group consisting of carbon dioxide, nitrogen, helium, neon and argon.

4. The process of claim 1 wherein the non-oxidizing material is nitrogen.

5. The process of claim 1 wherein the non-oxidizing material is carbon dioxide.

6. The process of claim 1 wherein palladium comprises from about 0.001 to about 0.028 weight percent of the catalyst, based on the total weight of the catalyst.

7. The process of claim 1 wherein the catalyst further comprises a metallic additive selected from the group consisting of silver, tin, copper, gold, lead, thallium, bismuth, cerium and alkali metals.

8. The process of claim 1 wherein the catalyst further comprises a metallic additive selected from the group consisting of silver, gold, thallium and alkali metals.

9. The process of claim 1 wherein the catalyst material further comprises silver as an additive.

10. The process of claim 9 wherein the catalyst material comprises from about 0.01 to about 0.02 weight percent palladium, from about 0.04 to about 0.15 weight percent of silver, wherein the ratio of the silver to the palladium is from about 1:1 to about 20:1, and wherein the weight percentages are based on the total weight of the prereduced catalyst.

11. The process of claim 1 wherein the selective hydrogenation process comprises a front-end hydrogenation process.

12. The process of claim 1 wherein the selective hydrogenation process comprises a tail-end ethylene purification process.

13. The process of claim 1 wherein the feed stock comprises a C₂ and C₃ olefinic feed stock.

14. A catalyst prepared by the process of claim 1.

15. A process for the selective hydrogenation of a feed stream comprising
preparing a hydrogenation catalyst by the process of claim 1,
without further reducing the catalyst, passing a selective hydrogenation feed stream over the catalyst in a selective hydrogenation process.

16. The process of claim 15 wherein the temperature of the feed stream is from about 35° C. to about 100° C.

17. The process of claim 15 wherein the catalyst further comprises a metallic additive selected from the group consisting of silver, gold, tin, lead, thallium, bismuth, cerium and alkali metals.

18. The process of claim 15 wherein the catalyst further comprises a metallic additive selected from the group consisting of silver, gold, thallium and alkali metals.

19. The process of claim 15 wherein the catalyst material further comprises silver as an additive.

20. The process of claim 15 wherein the selective hydrogenation process comprises a front-end hydrogenation process.

21. The process of claim 15 wherein the selective hydrogenation process comprises a tail-end ethylene purification process.
